(19) United States
(12) Patent Application Publication
(10) Pub. No.: US 2007/0219609 A1
(43) Pub. Date: Sep. 20, 2007

(54) IMPLANTABLE FIXATION ELEMENT

(75) Inventors: Peter Van Der Kemp, Heenvliet (NL); Simon Alexander Verheul, Nijmegen (NL); Kamiel Reinier Zale Geenen, Nijmegen (NL)

Correspondence Address: TROUTMAN SANDERS LLP, 600 PEACHTREE STREET , NE, ATLANTA, GA, 30308, US

(73) Assignee: Cardiall B.V., Varsseveld (NL)

(21) Appl. No.: 11/576,170

(22) PCT Filed: Sep. 14, 2005
(86) PCT No.: PCT/NL05/00670

§ 371 Date: Mar. 28, 2007

(30) Foreign Application Priority Data
Sep. 28, 2004 (IL) 1027131

(51) Int. Cl. A61N 1/05

(52) U.S. Cl. 607/149

(57) ABSTRACT

The invention provides an implantable fixation element for anchoring an elongate, for instance essentially cylindraical, element, particularly an electrical lead for a pacemaker in human or animal tissue, comprising a pair of first gripper beaks, movable to and from each other to enclose this element and, interconnected therewith, a pair of second fixation gripper beaks which, together with the first pair can hinge around a common point of rotation for a simultaneous opening and closing thereof, and having an open configuration with opposite ends, of which said second gripper beaks, the ends during the closing thereof, are adapted to penetrate the tissue, the beaks being provided with means to lock them together in the closed state. The invention also provides a sleeve to be used in combination with such a fixation element and an applicator, particularly intended to fix, by means of such a fixation element, the lead of a pacemaker to human or animal tissue.

The invention relates to an implantable fixation element for anchoring at least one elongate, for instance essentially cylindrical element in human or animal tissue, comprising a pair of first gripper beaks, movable to and from each other so as to enclose this element, and a fixing part, interconnected therewith and destined for engaging the tissue.

Such a fixation element is known from the published US Patent Application US2003/0,220,678 A1. This known fixation element, which is made from spring steel, comprises two beaks of which the respective ends cross each other and merge into the legs of a hairpin spring, so that the beaks are biased with a certain force towards each other.

This fixation element is used for anchoring flexible elongated and insulated electrical leads to the wall of a stomach; the free ends of the leads come to lie against the wall of the stomach and are connected to a suitable source of electrical stimulating pulses. To maintain the correct position of the ends a first fixation element is placed around the pair of leads and in the stomach, lying against the inner wall of the stomach while a second, similar fixation element, also lying against the outer wall of the stomach, is placed around the pair of leads but outside the stomach. In this way the electrodes are anchored there where the enter the stomach.

The problem of fixing insulated leads which carry electrical stimulating pulses to the surrounding tissue also occurs in the technology of pacemakers, implantable defibrillators and implantable cardioverters. There a supple, electrical lead is inserted by means of a catheter and through a vein, in most cases the vein which lies beneath the collar bone, and is then inserted into the heart for supplying the stimulating pulses. This electrical lead must be anchored to the surrounding tissue in de vicinity of the place where the lead exits from the vein. Although in the past, when pacemakers were firstly introduced, the surgeon used to fix the lead very simply by means of surgical stitching wire to the breast muscle, involving many risks, particularly the risk of destroying the insulation of the lead (which has, of course, disastrous results for the correct operation of a pacemaker, a defibrillator or a cardioverter), presently this anchoring is commonly done using a special protecting sleeve around the lead which is provided with circumference grooves, and this protecting sleeve is then fixed to the surrounding tissue by the surgeon by means of surgical stitching wire. This technique is described in PACE, vol. 15, November 1992, page 2005-2010, PACE, vol. 21, March 1998, page 549-552 and in the publication “A practical approach to permanent pacemaker implantation”, Peter H. Belott, M.D., Futura Publishing Company, Armonck, N.Y., USA, 1995 (ISBN 087973-594-4).

This procedure is, however, not an ideal one and obtaining a correct fixation is very much dependent upon the skill of the surgeon. Even when one uses the abovementioned protecting sleeve the final fixation is done by means of stitching wire and surgical knots, thus by manipulation, during which errors can be made, particularly by an incorrect tensioning of the wire (too tight or too loose), or by using incorrect knots.

Theoretically it could be possible to use the fixation element as known from the abovementioned US Patent Application US2003/0,220,678 A1 to anchor the electrical leads as used with a cardial pacemaker to the surrounding tissue but in that case, too, the surgeon must anchor the fixation device by means of stitching wire to the surrounding tissue—with all the drawbacks which this implies—, while, to obtain a correct fixation, the beaks of the fixation device must exercise a quite strong force on the lead which they enclose. As a result of the fact that the fixation device must be made from spring steel to obtain a sufficient pressure there is a very great risk of damage to the insulation of the leads, so that the metal can contact the electrically conducting core thereof, with damaging and, possibly fatal results.

The invention aims to obviate the above described drawbacks and particularly to provide a fixation element to be made from plastics which in a simple way and with one single operation encloses both the cylindrical element and anchors this to the surrounding tissue. This object is achieved according to the invention in that this fixating part comprises at least a pair of second fixation gripper beaks, capable for movement together with the first pair around a common point of rotation for a simultaneous opening and closing thereof and having an open configuration with opposite ends, of which second gripper beaks the ends, during closing, and capable to penetrate the tissue while means are provided to lock the beaks with respect to each other in the closed state.

As a result of the forced and simultaneous movement to each other of both the gripper beaks intended to surround the element to be anchored as well as the fixation grippers which enclose with their sharp ends the tissue it is ensured that in one step the element is enclosed reliably while at the same time this element is reliably anchored to the surrounding tissue.

An advantageous embodiment is obtained when the first and second pair of gripper beaks lie at the same side of a common point of rotation.

An embodiment which can be easily fabricated in an advantageous way is one in which the respective gripper beaks of the first and the second pair respectively are each formed by a first and second respectively hinge arm, hingedly interconnected at one end, each such hinge arm having, starting from the hinge point, a first part-circular cut-out, said first cut-outs defining together the pair of first gripper beaks, with adjoining a second part-circular cut-out, said second cut-outs defining together the second pair of fixation gripper beaks, while, between the first and second cut-outs respectively, interacting catching coupling parts are provided.

Preferably the two hinge arms are made in one piece and each hinge point is made up by a thin strip of flexible material.

Preferably each hinge arm is at its outer side provided with a out-cut to cooperate with a applicator excerting an inwardly directed force thereon.

The claimed exclusive rights include an applicator for attaching a fixation element such as described in claim 6.

A fixation element can be connected detachably to a part of this applicator, such as described in claim 7, resulting into a disposable unit which can be sterilized as such.

The claimed exclusive rights also include a sleeve such as subject of claim 8.

It is observed that staples for surgical uses, of which the legs have, in cross-section, a C-shaped configuration and of which the points can penetrate the tissue when a force is excerted on the legs are known in itself, for instance from the U.S. Pat. Nos. 5,242,457 and 5,618,311, the US patent application 2003/0,111,641 A1 and the European patent application 0 565 892 A1. Applicators for surgical staples are also known—vide for instance the U.S. Pat. No. 6,059,799 and the International patent application WO99/18857.

U.S. Pat. No. 4,144,890 discloses a contact device for muscle stimulation, particularly an epicardial electrode, having a pair of spaced-apart prong-like attachment members, and also discloses an applicator tool for positioning same. The use of prong-like attachment members on an epicardial electrode is furthermore disclosed in US 2004/0 176 830 A1,as well as in U.S. Pat. No. 4,142,530 which describes an epicardial lead with an electrode tip provided with such members.

However, none of these publications discloses the subject of the present application, while this subject also cannot be considered to be obvious after having taken notice of these publications.

The invention is elucidated on the hand of the drawing.

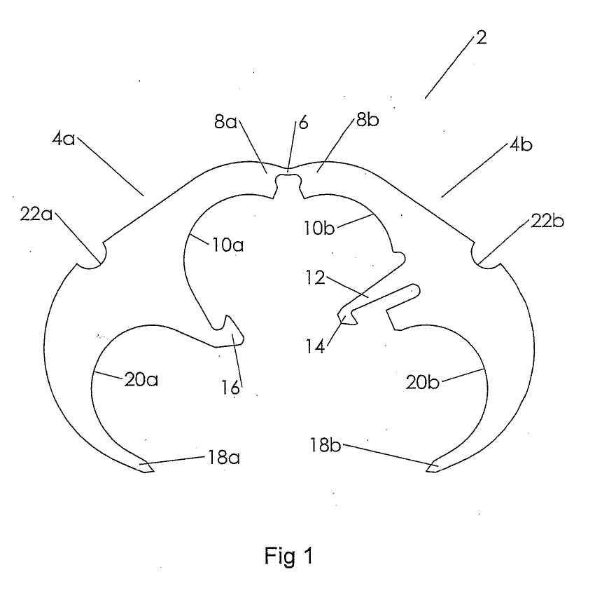

FIG. 1 shows on a very enlarged scale a front view of a fixation element according to the invention in the open state.

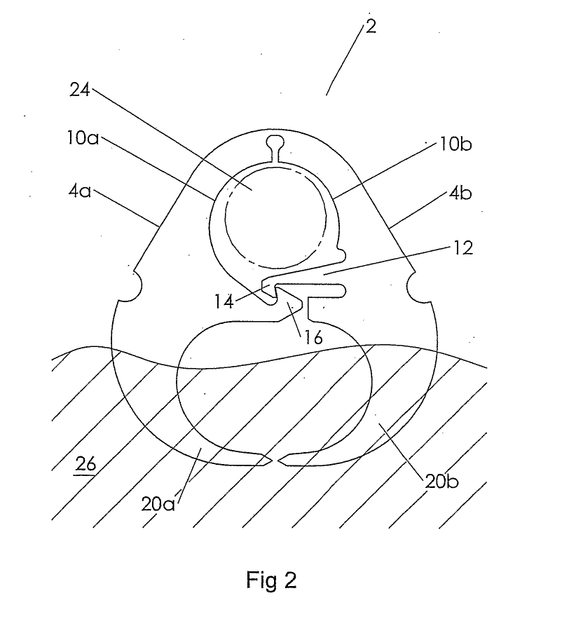

FIG. 2 shows a front view of this element, now in the locked, and closed state.

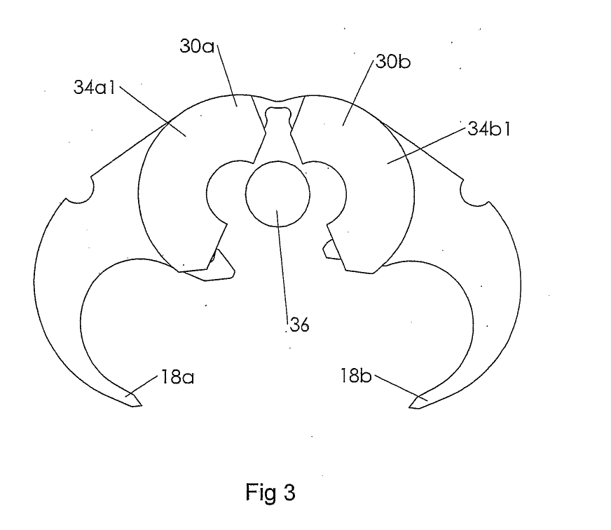

FIG. 3 shows a front view corresponding to the view according to FIG. 1, but now of the fixation element combined with a, protecting sleeve preferably to be used therewith, and with a cylindrical element to be fixed therewith.

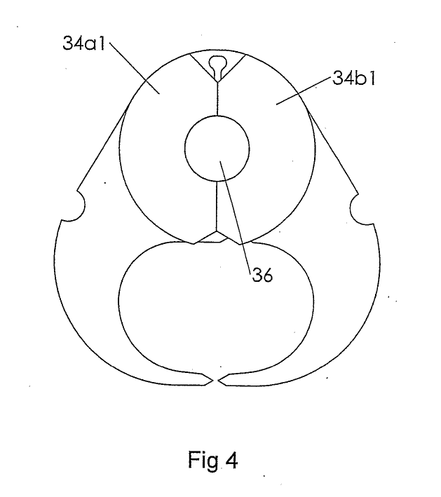

FIG. 4 shows a similar front view with the fixation element in the closed state.

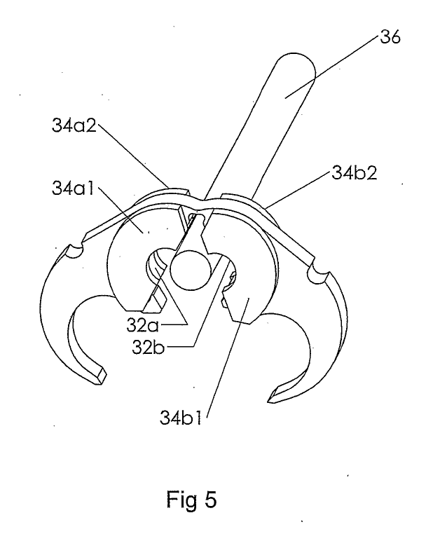

FIG. 5 is a perspective view of the combination: fixation element, protecting sleeve and element to be enclosed thereby.

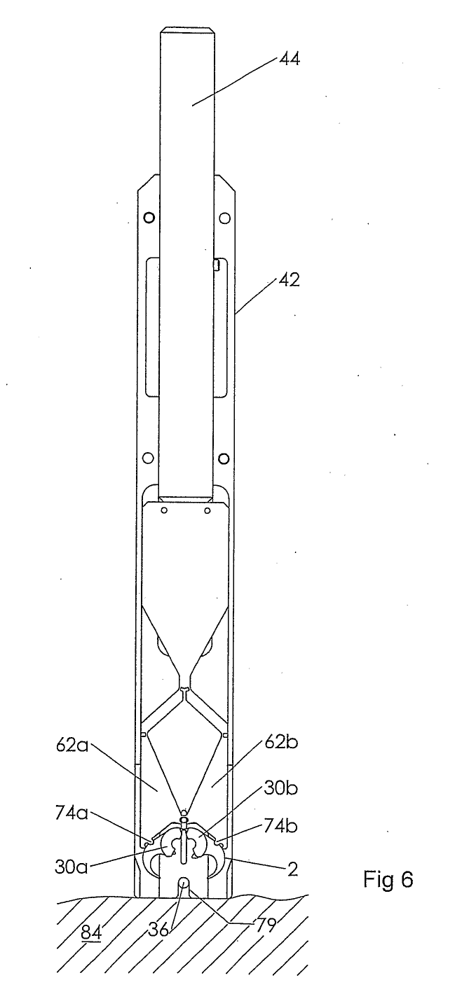

FIG. 6-10 are front views of the principle parts of an applicator to be used in combination with the fixation element in the various operative states thereof.

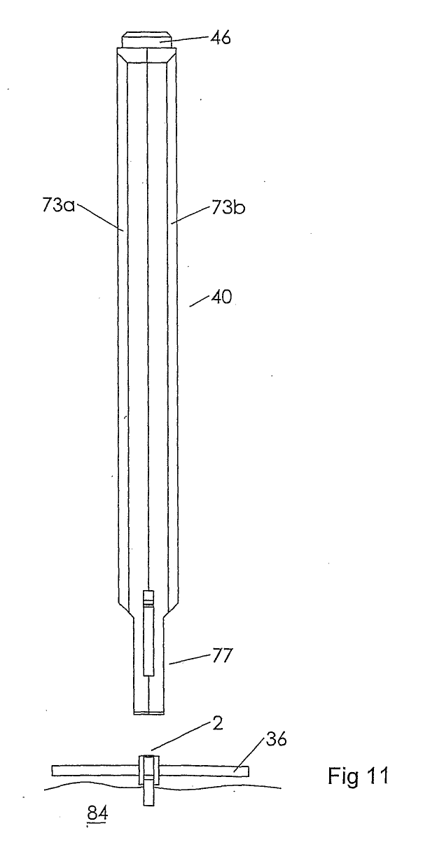

FIG. 11 shows a side view of an applicator with, shown there beneath, an element fixed to tissue.

In FIG. 1 the fixation element is indicated in its entirety by reference numeral 2; it is made in one piece, for instance by molding, from a suitable plastics and comprises a first arm 4a and a second arm 4b,of which the respective ends 8a, 8b are interconnected hingedly by means of a thin strip of material 6. Both the arms have a first partly circular cut-out 10a, 10b, lying close to the interconnecting point 6, with directly there under a locking mechanism comprising a biased hooking arm 12, protruding from the arm 4b, with the cross-hook 14, cooperating with and a take-up hook 16, protruding from the arm 4a. The arms 4a, 4b end in pointed teeth 18a, 18b and there is, between the tooth 18a and the take-up hook 16 as well as between the tooth 18b and the locking hook 12 a second partly circular cut-out, indicated with 20a and 20b, respectively.

Both the arm 4a and the arm 4b has at the outer circumference a cut-out 22a and 22b, respectively; the purpose thereof will be described later on.

FIG. 2 shows the fixation element in the closed state, in which the arms 4a, 4b are moved towards each other and the hook end 14 hooks around the take-up hook 16. It is clear that the cut-outs 10a, 10b in combination with the hookarm 12 define between them an essentially cylindrical space in which a schematically indicated element 24, here shown as an elongate circelcylindrical element , can be enclosed while, when the pointed teeth 18a, 18b during the closing of the fixation element had rested with some force on the upper surface of a tissue 26, these teeth have by now penetrated this tissue to a certain depth. In this way the fixation element is anchored to the tissue and the cylindrical element 24, too, is anchored to this tissue.

In the medical practice and certainly when the fixation element is used to anchor to tissue electrical leads which carry electrical pulses (said lead thus being the cylindrical element to be anchored) one will preferably position between this lead and the fixation element a protecting sleeve, such as shown in the drawings 3 to and including 5. FIG. 3 shows such a sleeve which comprises two halves 30a, 30b and each halve—see in particular FIG. 5—comprises a central hub-shaped part 32a, 32b, bounded by to end flanges 34a1, 34a2 and 34b1, 34b2, respectively. The space with a cylindrical cross-section as defined by the hub parts 32a, 32b accommodates the cylindrical element which is to be anchored, thus the flexible insulated lead 36 carrying the electrical pulses. It will be clear from the preceding that the fixation element will be attached to the tissue in one operation, namely the movement towards each other of the arms 4a and 4b until the locking elements are intercoupled and the entirety is brought into the closed state, in which the element to be anchored is reliably surrounded and anchored to the tissue in the desired position.

Exercizing the closing forces is preferably done at the position of the cut-outs 22a, 22b in the arms 4a, 4b and a suitable applicator according to the invention by means of which this can be brought about will be elucidated on the hand of FIGS. 6 to and including 11.

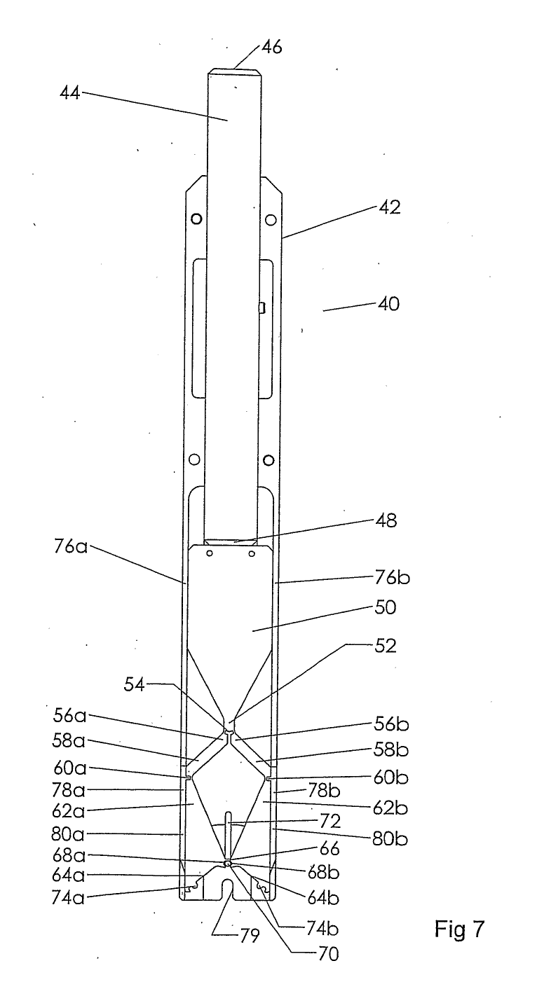

These figures show the parts of the applicator, which is in its entirety indicated with reference numeral 40—see the FIGS. 6 and 7. These parts are accommodated in an elongated prismatic housing 42 with a greater dimension in the plane of the drawing (the width), then in a direction perpendicular to the plane of the drawing (the thickness). The simple structure of this applicator makes a cheap production thereof possible so that it can be discarded after use.

The housing 42 accommodates slidingly an elongate pressure pin 44 of which during use the upper end 46 must be pressed downwardly and of which the lower end 48 bears on a sliding actuator 50; the lower end 52 thereof is hingedly (at the position 54a, 54b) connected to the upper ends 56a, 56b of two pressure arms 58a, 58b, the narrowing lower ends 60a, 60bthereof merge into two triangular pressure pieces 62a, 62b, each having the shape of an obtuse triangle with long sides and a short basis, 64a, 64b. The obtuse points thereof are, at 66, interconnected and beneath this interconnecting point cut-outs 68a, 68b enclose a guide pin 70. This guide pin 70 is thus perpendicular to the plane of the drawing and is guided in two narrow, guiding slits 72, which lie in the longitudinal axis of symmetry and are provided in the front and rear wall (perpendicular to the plane of the drawing) of the applicator 40. From each basis 64a, 64b protudes a cam indicated with 74a, 74b, respectively, intended for cooperation with a cut-out 22a, 22b of a fixation element which is to be applicated. The front wall 73a and the rear wall 73b of the housing are at the narrowed lower end 77 provided with an entry slit 79, by means of which the applicator is placed around a cylindrical element 76 which is to be put into place while furthermore each of the sidewalls 76a, 76b has an elongated cut-out 78a, 78b, the purpose of which will be described later on.

It is observed that the actuator 40, the arms 58a, 58b, interconnected therewith and the two triangular pressure pieces 62a, 62b can be made up in one piece from a suitable plastics, while the hinges 60a, 60b and 68a, 68b consist of parts with a small thickness of the material, so that there a so-called “bending hinge” is obtained.

FIG. 6 shows the initial position in which the pressure pin 44 protrudes as far as possible from the housing 42, the triangular pressure pieces 62a, 62b have their upper position and the distance between the cams 74a, 74b is at its maximum. In this state an—open—fixation element 2 can be supplied into the housing 42, said fixation element 2 being provided with the two sleeve halves 30a, 30b (see FIG. 3). In this state the housing 42 is placed with the feeding slit 79 around the cylindrical element 36 which is to be anchored, with the lower end of the housing resting upon the tissue 84 to which the anchoring must take place.

FIG. 7 shows the respective positions of the various parts in the state in which, by pressing the end of the pressure pin 74, through the actuator 50, the upper ends 56a, 56b of the arms 58a, 58b have just narrowed at the upper ends of the cut-outs 78a, 78b and the hinge pin 70 lies at the lower end of the guide 72. For clarity reasons in this drawing the fixation element 2 is not shown.

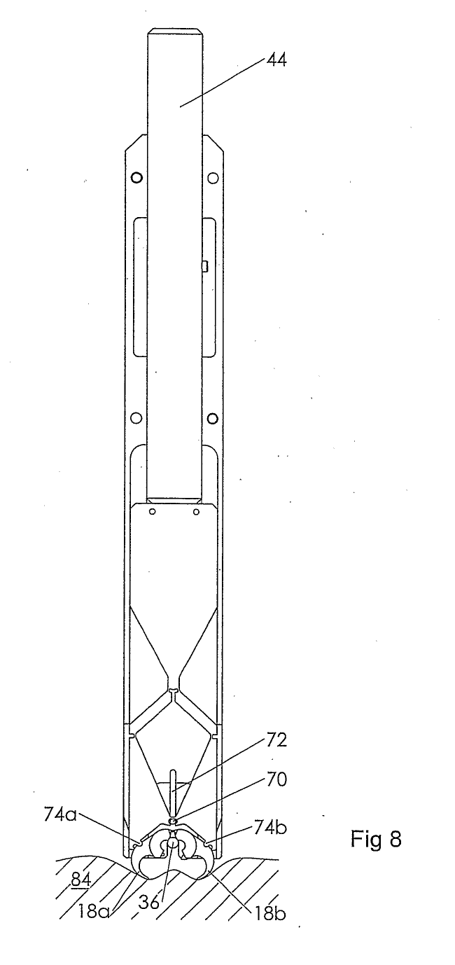

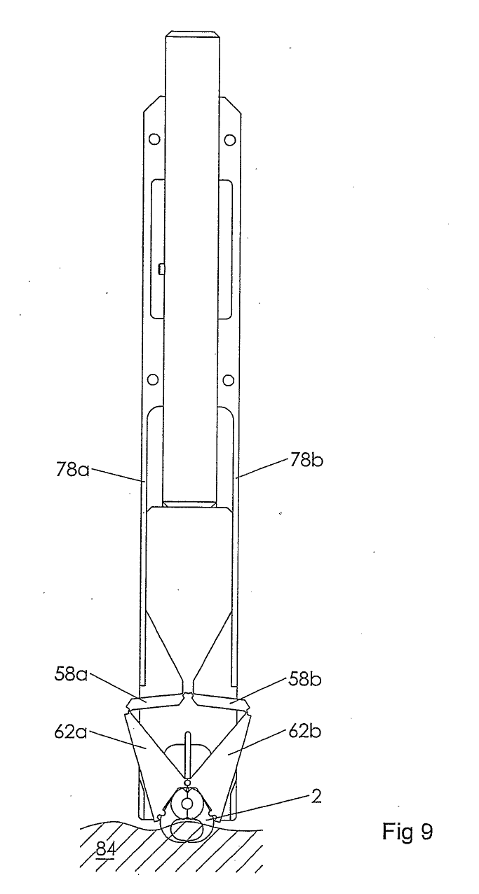

FIG. 8 and 9 show how, by pressing the pin 44, the parts have now reached their lowest position, in which the teeth 18a, 18b rest on the tissue 84. The fixation element however is not yet closed because the mutual distance of the cams 74a, 74b has not changed. The pin 70 has now arrived at the lower end of the guiding slid 72 and as a result the hinge point of the pressure pieces 62a, 62b cannot move further downwardly.

A further downwardly pressing of the pin 77 results into the situation of FIG. 9. As the hinge points 60a, 60b between the arm 58a and pressure piece 62a on the one hand, and arm 58b and pressure piece 62b on the other hand now lie there where the cut-outs 78a, 78b in the respective sidewalls 76a, 76b begin, the arms 58a, 58b can move outwardly to the left and right respectively in the plane of the drawing, they move the points of the triangular pressure pieces 62a, 62b with hem in this movement and thus decrease the distance between the cams 74a, 74b. Thus the fixation element 2 is closed while the teeth 18a, 18b thereof penetrate into the underlying tissue.

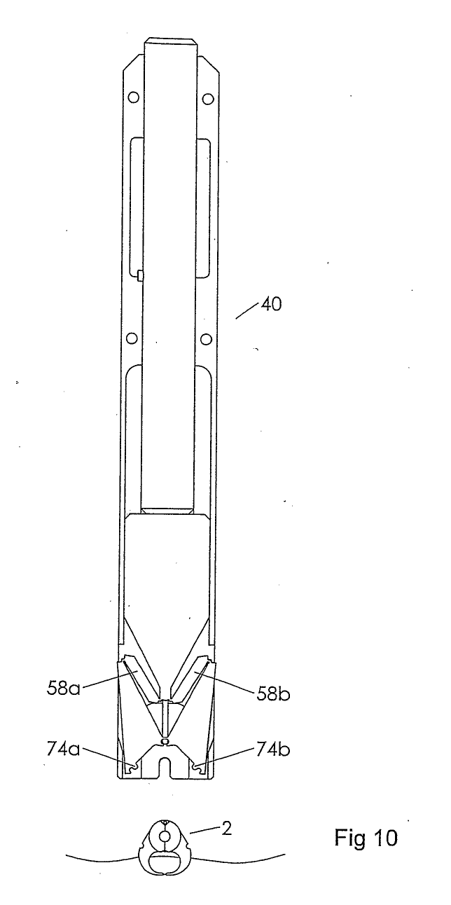

When the pin 44 is pressed downwardly still further the situation according to FIG. 10 is reached. The arms 58a, 58b are now entirely folded inwardly and the cams 74a, 74b have by now moved away from each other; the applicator 40 can now be taken off, leaving in the tissue 84 the fixation element 2 with the cylindrical element 36 (in particular a pacemaker guide) enclosed thereby such as shown in FIG. 10 in front view and in FIG. 11 in side view.

It is clear that the application is not limited to the described embodiment thereof; many other uses are possible.

Particularly in the fixation element the shape of the accommodating space can differ from the one which is shown in the drawings, for instance to accommodate an element with a non circular cross-section. Furthermore a fixation element can be interconnected with one of the pressure pieces by means of at least one connection to be ruptured so that it is not necessary to position a loose fixation element in the applicator and as a result one obtains a complete apparatus which is to be used only once. Furthermore two or even more fixation elements can be interconnected and put into place in one operation.

1. Implantable fixation element for anchoring at least one elongate element in human or animal tissue, comprising: a pair of first gripper beaks, movable to and from each other so as to enclose the element, and a fixing part, interconnected therewith and destined for engaging the tissue, wherein the fixing part comprises at least a pair of second fixation gripper beaks, capable for movement together with the first pair around a common point of rotation for a simultaneous opening and closing thereof and having an open configuration with opposite ends, of which second gripper beaks the ends, during closing, and capable to penetrate the tissue while means are provided to lock the beaks with respect to each other in the closed state.

2. Fixation element according to claim 1, wherein the first and second pair of gripper beaks lie at the same side of the common point of rotation.

3. Fixation element according to claim 2, wherein the respective gripper beaks of the first and the second pair respectively are each formed by a first and second respectively hinge arm, hingedly interconnected at one end, each such hinge arm having, starting from the hinge point, a first part-circular cut-out, said first cut-outs defining together the pair of first gripper beaks, with adjoining a second part-circular cut-out, said second cut-outs defining together the second pair of fixation gripper beaks, while, between the first and second cut-outs respectively, interacting catching coupling parts are provided.

4. Fixation element according to claim 3, wherein the two hinge arms are made in one piece and each hinge point is made up by a thin strip of flexible material.

5. Fixation element according to claim 3, wherein each hinge arm is at its outer side provided with a cut-out to cooperate with an applicator exercising an inwardly directed force thereon.

6. Applicator to be used in combination with a fixation element according to claim 3, comprising an elongate housing with a operating element, slidable in the longitudinal direction of the housing and coupled to an actuator, also slidable in the longitudinal direction of the housing, said actuator acting with a central part on the connecting point of the first ends of two pressure arms which include an obtuse angle and of which second ends, which lie in a direction perpendicular to the house axis at a distance of each other can move outwardly through cut-outs in the housing walls and, which each exert a pressure on a tip of an essentially triangular pressure piece of which the respective obtuse basis points are hingedly interconnected and are, movable over a limited distance, guided in the housing in the longitudinal direction thereof, each of said pressure pieces being provided, next to the other basis point for cooperation with a fixation element to be applicated, while the lower end of the housing has a space for accommodating a fixation element to be applicated.

7. Fixation element according to claim 1, wherein it is, by means of a rupture connection, connected with at least one of the pressure pieces of the applicator according to claim 6.

8. Sleeve to be used in combination with a fixation element according to claim 1, comprising two joining halves, each with a central cylindrical body part with an inner bore adapted to the diameter of an element to be accommodated therein, and with two radial flanges with a mutual distance adapted to the thickness of the gripper beaks of the fixation element.

9. Fixation element according to claim 4, wherein each hinge arm is at its outer side provided with a cut-out to cooperate with an applicator exercising an inwardly directed force thereon.

10. Fixation element according to claim 3, wherein it is, by means of a rupture connection, connected with at least one of the pressure pieces of the applicator according to claim 6.

11. Sleeve to be used in combination with a fixation element according to claim 3, comprising two joining halves, each with a central cylindrical body part with an inner bore adapted to the diameter of an element to be accommodated therein, and with two radial flanges with a mutual distance adapted to the thickness of the gripper beaks of the fixation element.
